(19) United States
(12) Patent Application Publication
(10) Pub. No.: US 2021/0292723 A1
(43) Pub. Date: Sep. 23, 2021

(54) Alpha-Amylase Variants and Polynucleotides Encoding Same

(71) Applicant: Novozymes A/S, Bagsvaerd (DK)

(72) Inventors: Thomas Gibbons, Wake Forest, NC (US); Carsten Andersen, Vaerloese (DK); Prahlada Vasudeva Rao, Bangalore (IN)

(73) Assignee: Novozymes A/S, Bagsvaerd (DK)

(21) Appl. No.: 16/347,537

(22) PCT Filed: Nov. 29, 2017
(86) PCT No.: PCT/US2017/063603

§ 371 Date: May 3, 2019

Related U.S. Application Data
(60) Provisional application No. 62/427,665, filed on Nov. 29, 2016.

(51) Int. Cl. C12N 9/28

(52) U.S. Cl. CPC C12N 9/2417; C12Y 302/01001

(57) ABSTRACT

The present invention relates to alpha-amylase variants. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to alpha-amylase variants, polynucleotides encoding the variants, methods of producing the variants, and methods of using the variants.

### Description of the Related Art

Alpha-amylases (E.C. 3.2.1.1) constitute a group of enzymes which catalyze hydrolysis of starch, glycogen and related polysaccharides and oligosaccharides.

Alpha-amylases are used commercially for a variety of purposes such as in the initial stages of starch processing (e.g., liquefaction); in wet milling processes; and in alcohol production from carbohydrate sources. They are also used as cleaning agents or adjuncts in detergent matrices; in the textile industry for starch desizing; in baking applications; in the beverage industry; in oil fields in drilling processes; in recycling processes, e.g., for de-inking paper; and in animal feed.

Some commercial alpha-amylases for, e.g., starch liquefaction originate from Bacillus licheniformis or Bacillus stearothermophilus. Protein engineered variants of wild type enzymes have been developed to overcome process issues. There is still a need, though, for novel alpha-amylases with improved properties, such as higher stability at low pH, low calcium and high temperature. Such enzymes will allow the starch liquefaction process to be run at reduced pH which has a positive influence on chemical savings.

It is an object of the present invention to provide novel alpha-amylase variants having an increased stability at low pH and/or at high temperature, in particular at low calcium concentrations.

For an alpha-amylase to be used in a starch liquefaction process it is of particular interest that it is thermostable and able to function at low pH and low calcium concentrations. Altered Ca²⁺ stability means the stability of the enzyme under Ca′ depletion has been improved, i.e., higher or lower stability. In the context of the present invention, mutations (including amino acid substitutions) of importance are mutations achieving altered Ca²⁺ stability, in particular improved Ca²⁺ stability, i.e., higher or lower stability, at especially low pH (i.e., pH 4-6).

WO2000/060059 disclose Termamyl like alpha-amylase variants having increased stability at low Ca²⁺ levels. WO2013/057143 and WO2013/057141 disclose variants of alpha-amylases from Bacillus liquefaciens having improved properties such as increased stability at low calcium concentrations. The present invention provides alpha-amylase variants with improved properties compared to its parent.

### SUMMARY OF THE INVENTION

The present invention relates to an alpha-amylase variant comprising a substitution at a position corresponding to position 116, 393, 181, 293, 264, or 196 of SEQ ID NO: 1, in particular one or more substitutions selected from the group consisting of T116N, T116D, T116E, T116Q, T116Y, T181H, T181Q, S264K, L196D, H293Q, and Q393L, wherein the variant has at least 75%, at least 80%, at least 85%, at least 90%, at least 95%, at least 96%, at least 97%, at least 98%, or at least 99%, but less than 100% sequence identity to amino acids 1 to 483 of SEQ ID NO: 1, or amino acids 1 to 481 of SEQ ID NO: 2; and wherein the variant has alpha-amylase activity. The present invention also relates to polynucleotides encoding the variants; nucleic acid constructs, vectors, and host cells comprising the polynucleotides, and to compositions comprising the variants of the invention; and methods of producing the variants.

The present invention also relates to a process for producing a syrup from starch-containing material comprising the steps of:

a) liquefying the starch-containing material at a temperature above the initial gelatinization temperature in the presence of a variant alpha-amylase according to any of the claims 1-10; and
b) saccharifying the product of step a) in the presence of a glucoamylase.

### Definitions

Alpha-amylase variants: Alpha-amylases (E.C. 3.2.1.1) are a group of enzymes which catalyze the hydrolysis of starch and other linear and branched 1,4 glucosidic oligo- and polysaccharides. The skilled person will know how to determine alpha-amylase activity. It may be determined according to the procedure described in the Examples, e.g., by the PNP-G7 assay or the EnzCheck assay. In one aspect, the variants of the present invention have at least 20%, e.g., at least 40%, at least 50%, at least 60%, at least 70%, at least 80%, at least 90%, at least 95%, or at least 100% of the alpha-amylase activity of the polypeptide of SEQ ID NO: 3. In one aspect, a variant of the present application has at least 20%, e.g., at least 40%, at least 50%, at least 60%, at least 70%, at least 80%, at least 90%, at least 95%, or at least 100% of the alpha-amylase activity of its parent. In a further embodiment the variant alpha-amylases of the invention have an increased stability compared to a parent alpha-amylase, particularly the parent disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. The residual activity is in one embodiment at least 45%, at least 50%, particularly at least 55%.

In another embodiment the variant alpha-amylases of the invention are capable of generating a liquefact having a dextrose equivalent (DE) value higher than the DE value generated by a parent alpha-amylase, particularly the parent disclosed as amino acids 1 to 481 of SEQ ID NO: 2. In particular the DE value is at least 2 higher than the DE value generated by the parent alpha amylase of SEQ ID NO: 2.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has alpha-amylase activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such improved properties include, but are not limited to, increased stability measured as residual alpha-amylase activity determined by EnzCheck assay after 15 min incubation at 90° C., pH 4.5, 5 ppm Ca²⁺; and/or the variants are capable of generating a liquefact having a dextrose equivalent (DE) value higher than the DE value generated by a parent alpha-amylase.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 483 of SEQ ID NO: 1, or amino acids 1 to 481 of SEQ ID NO: 2.

Mature polypeptide coding sequence: The term “mature polypeptide coding sequence” means a polynucleotide that encodes a mature polypeptide having alpha-amylase activity.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent alpha-amylase: The term “parent” or “parent alpha-amylase” means any polypeptide with alpha-amylase activity to which an alteration is made to produce the enzyme variants of the present invention.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Variant: The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the alpha-amylase activity of the parent alpha-amylase. In one embodiment the parent alpha-amylaase is selected from the polypeptide of SEQ ID NO: 1, 2, or 3.

Wild-type alpha-amylase: The term “wild-type” alpha-amylase means an alpha-amylase expressed by a naturally occurring microorganism, such as a bacterium, yeast, or filamentous fungus found in nature.

### Conventions for Designation of Variants

For purposes of the present invention, the polypeptide disclosed in SEQ ID NO: 1 is used to determine the corresponding amino acid residue in another alpha-amylase. The amino acid sequence of another alpha-amylase, e.g., the variants according to the invention, is aligned with the polypeptide disclosed as SEQ ID NO: 1, and based on the alignment, the amino acid position number corresponding to any amino acid residue in the polypeptide disclosed as SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537:39-64; Katoh and Toh, 2010, Bioinformatics 26:1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple Alterations.

Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different Alterations.

Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg 170Gly”, “Tyr167Gly+Arg 170Ala”, “Tyr167Ala+Arg170Gly”, and “Tyr167Ala+Arg170Ala”.

### DETAILED DESCRIPTION OF THE INVENTION

The present invention relates to alpha-amylase variants comprising a substitution at a position corresponding to position 116, 393, 181, 293, 264, or 196 of SEQ ID NO: 1, wherein the variant has at least 75%, at least 80%, at least 85%, at least 90%, at least 95%, at least 96%, at least 97%, at least 98%, or at least 99%, but less than 100% sequence identity to amino acids 1 to 483 of SEQ ID NO: 1, or amino acids 1 to 481 of SEQ ID NO: 2; and wherein the variant has alpha-amylase activity.

In one embodiment the variants are isolated.

### Variants

The present invention provides alpha-amylase variants, comprising a substitution at one or more (e.g., several) positions corresponding to positions 116, 393, 181, 293, 264, and 196 of SEQ ID NO: 1, wherein the variant has alpha-amylase activity.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent alpha-amylase.

In one embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to amino acids 1 to 483 of SEQ ID NO: 1.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to amino acids 1 to 481 of SEQ ID NO: 2.

In one aspect, the number of alterations in the variants of the present invention is 1-20, e.g., 1-10 and 1-5, such as 1, 2, 3, 4, 5, 6, 7, 8, 9 or 10 alterations.

In another aspect, a variant comprises a substitution at one or more (e.g., several) positions corresponding to positions 116, 393, 181, 293, 264, and 196 of SEQ ID NO: 1. In another aspect, a variant comprises a substitution at two positions corresponding to any of positions 116, 393, 181, 293, 264, and 196 of SEQ ID NO: 1. In another aspect, a variant comprises a substitution at three positions corresponding to any of positions 116, 393, 181, 293, 264, and 196 of SEQ ID NO: 1.

In another aspect, the variant comprises or consists of a substitution at a position corresponding to position 116 of SEQ ID NO: 1. In another aspect, the amino acid at a position corresponding to position 116 is substituted with Ala, Arg, Asn, Asp, Cys, Gln, Glu, Gly, His, Ile, Leu, Lys, Met, Phe, Pro, Ser, Trp, Tyr, or Val, preferably with Asn, Asp, Gln, or Glu. In another aspect, the variant comprises or consists of the substitution T116N,D,Q,E,Y using the polypeptide of SEQ ID NO: 1 for numbering.

In another aspect, the variant comprises or consists of a substitution at a position corresponding to position 181 of SEQ ID NO: 1. In another aspect, the amino acid at a position corresponding to position 181 is substituted with Ala, Arg, Asn, Asp, Cys, Gln, Glu, Gly, His, Ile, Leu, Lys, Met, Phe, Pro, Ser, Trp, Tyr, or Val, preferably with Gln, or His. In another aspect, the variant comprises or consists of the substitution T181Q,H using the polypeptide of SEQ ID NO: 1 for numbering.

In another aspect, the variant comprises or consists of a substitution at a position corresponding to position 196 of SEQ ID NO: 1. In another aspect, the amino acid at a position corresponding to position 196 is substituted with Ala, Arg, Asn, Asp, Cys, Gin, Glu, Gly, His, Ile, Lys, Met, Phe, Pro, Ser, Thr, Trp, Tyr, or Val, preferably with Asp. In another aspect, the variant comprises or consists of the substitution L196D using the polypeptide of SEQ ID NO: 1 for numbering.

In another aspect, the variant comprises or consists of a substitution at a position corresponding to position 264 of SEQ ID NO: 1. In another aspect, the amino acid at a position corresponding to position 264 is substituted with Ala, Arg, Asn, Asp, Cys, Gin, Glu, Gly, His, Ile, Leu, Lys, Met, Phe, Pro, Thr, Trp, Tyr, or Val, preferably with Lys. In another aspect, the variant comprises or consists of the substitution Q,S264K using the polypeptide of SEQ ID NO: 1 for numbering.

In another aspect, the variant comprises or consists of a substitution at a position corresponding to position 293 of SEQ ID NO: 1. In another aspect, the amino acid at a position corresponding to position 293 is substituted with Ala, Arg, Asn, Asp, Cys, Gin, Glu, Gly, Ile, Leu, Lys, Met, Phe, Pro, Ser, Thr, Trp, Tyr, or Val, preferably with Gin. In another aspect, the variant comprises or consists of the substitution H293Q using the polypeptide of SEQ ID NO: 1 for numbering.

In another aspect, the variant comprises or consists of a substitution at a position corresponding to position 393 of SEQ ID NO: 1. In another aspect, the amino acid at a position corresponding to position 393 is substituted with Ala, Arg, Asn, Asp, Cys, Glu, Gly, His, Ile, Leu, Lys, Met, Phe, Pro, Ser, Thr, Trp, Tyr, or Val, preferably with Leu. In another aspect, the variant comprises or consists of the substitution Q393L using the polypeptide of SEQ ID NO: 1 for numbering.

In another aspect, the variant comprises or consists of a substitution at positions corresponding to positions 116+181, such as those described above.

In another aspect, the variant comprises or consists of substitutions at positions corresponding to positions 116+196, such as those described above.

In another aspect, the variant comprises or consists of substitutions at positions corresponding to positions 116+264, such as those described above.

In another aspect, the variant comprises or consists of substitutions at positions corresponding to positions 116+293, such as those described above.

In another aspect, the variant comprises or consists of substitutions at positions corresponding to positions 116+393, such as those described above.

In another aspect, the variant comprises or consists of substitutions at positions corresponding to positions 181+196, such as those described above.

In another aspect, the variant comprises or consists of substitutions at positions corresponding to positions 181+264, such as those described above.

In another aspect, the variant comprises or consists of substitutions at positions corresponding to positions 181+293, such as those described above.

In another aspect, the variant comprises or consists of substitutions at positions corresponding to positions 181+393, such as those described above.

In another aspect, the variant comprises or consists of substitutions at positions corresponding to positions 196+264, such as those described above.

In another aspect, the variant comprises or consists of substitutions at positions corresponding to positions 196+293, such as those described above.

In another aspect, the variant comprises or consists of substitutions at positions corresponding to positions 196+393, such as those described above.

In another aspect, the variant comprises or consists of substitutions at positions corresponding to positions 264+293, such as those described above.

In another aspect, the variant comprises or consists of substitutions at positions corresponding to positions 264+393, such as those described above.

In another aspect, the variant comprises or consists of substitutions at positions corresponding to positions 293+393, such as those described above.

In another aspect, the variant comprises or consists of a substitution or combination of substitutions selected from T116N, T116D, T116Q, T116Y, Q,S264K, L196D, Q393L, and T116D+L196D (using SEQ ID NO: 1 for numbering).

In another aspect, the variant comprises or consists of a substitution or combination of substitutions selected from T116N, T116D, T116Q, T116E, T116Y, T181Q, T181H, Q,S264K, Q393L, and H293Q (using SEQ ID NO: 1 for numbering).

In a preferred embodiment the parent alpha-amylase is the one disclosed herein as SEQ ID NO: 2.

In one particular embodiment the present invention relates to a variant alpha-amylase, comprising a substitution at a position corresponding to position 116, 393, 264, or 196 of SEQ ID NO: 1, in particular one or more substitutions selected from the group consisting of T116N, T116D, T116Q, T116Y, S264K, L196D, Q393L, and T116D+L196D; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In another particular embodiment the present invention relates to a variant alpha-amylase, comprising a substitution at a position corresponding to position 116 of SEQ ID NO: 1, in particular one or more substitutions selected from the group consisting of T116N; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In another particular embodiment the present invention relates to a variant alpha-amylase, comprising a substitution at a position corresponding to position 116 of SEQ ID NO: 1, in particular one or more substitutions selected from the group consisting of T116D; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In another particular embodiment the present invention relates to a variant alpha-amylase, comprising a substitution at a position corresponding to position 116 of SEQ ID NO: 1, in particular one or more substitutions selected from the group consisting of T116Q; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In another particular embodiment the present invention relates to a variant alpha-amylase, comprising a substitution at a position corresponding to position 116 of SEQ ID NO: 1, in particular one or more substitutions selected from the group consisting of T116Y; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In another particular embodiment the present invention relates to a variant alpha-amylase, comprising a substitution at a position corresponding to position 116 and 196 of SEQ ID NO: 1, in particular substitutions selected from the group consisting of T116D+L196D; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca′. In particular the residual activity is at least 45%, at least 50%, such as at least 55%.

In one particular embodiment the present invention relates to a variant alpha-amylase, comprising a substitution at a position corresponding to position 264 of SEQ ID NO: 1, in particular a substitutions selected from Q,S264K; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In one particular embodiment the present invention relates to a variant alpha-amylase, comprising a substitution at a position corresponding to position 393 of SEQ ID NO: 1, in particular a substitutions selected from Q393L; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In one particular embodiment the present invention relates to a variant alpha-amylase, comprising a substitution at a position corresponding to position 196 of SEQ ID NO: 1, in particular a substitutions selected from L196D; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the variant has an increased stability compared to the parent alpha-amylase disclosed as amino acids 1 to 481 of SEQ ID NO: 2, and wherein the increased stability is measured as residual alpha-amylase activity determined by EnzCheck assay after 15 min incubation at 90° C., pH 4.5, 5 ppm Ca²⁺. In particular the residual activity is at least 45%, at least 50%, such as at least 55%.

In one particular embodiment the present invention relates to a variant alpha-amylase, comprising a substitution at a position corresponding to position 116, 181, 393, 264, or 293 of SEQ ID NO: 1, in particular one or more substitutions selected from the group consisting of T116N, T116D, T116Q, T116E, T116Y, T181H, T181Q, Q,S264K, H293Q, and Q393L; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variants are capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 116 of SEQ ID NO: 1, in particular the substitution T116N; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 116 of SEQ ID NO: 1, in particular the substitution T116D; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO:2, wherein the variant is capable of generating a liquefact having a dextrose equivalent (DE) value higher than the DE value generated by a parent alpha-amylase disclosed as amino acids 1 to 481 of SEQ ID NO:2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 116 of SEQ ID NO: 1, in particular the substitution T116Q; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 116 of SEQ ID NO: 1, in particular the substitution T116E; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 116 of SEQ ID NO: 1, in particular the substitution T116Y; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 181 of SEQ ID NO: 1, in particular the substitution T181Q; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 181 of SEQ ID NO: 1, in particular the substitution T181H; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 264 of SEQ ID NO: 1, in particular the substitution Q,S264K; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 393 of SEQ ID NO: 1, in particular the substitution Q393L;

wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In one particular embodiment the present invention relates to a variant alpha-amylase, comprising a substitution at position 293 of SEQ ID NO: 1, in particular the substitution H293Q; wherein the variant has at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, wherein the variant is capable of generating a liquefact having a dextrose equivalent (DE) value higher than the DE value generated by a parent alpha-amylase disclosed as amino acids 1 to 481 of SEQ ID NO: 2, particularly the DE value is at least 2 higher than the DE value generated by the parent alpha amylse of SEQ ID NO: 2, more particularly the DE value is in the range from 12.5 to 22, such as from 13 to 21.

In a further particular embodiment the present invention relates to a variant alpha-amylase comprising a substitution or combination of substitutions selected from:

### T116N;

### T116D;

### T116Q;

### T116E;

### T116Y;

### Q,S264K;

### L196D;

### Q393L; and

T116D+L196D (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+(Q264S);

### b) G48A+T49I+G107A+H156Y+A181T+N190F+L201F+A209V+(Q264S); or

### c) G48A+T49I+G107A+H156Y+A181T+N190F+L201F+A209V+H68W+K176L+E185P+F201Y+H205Y+K213T+Q360S+D416V+R437W+(Q264S).

In a further particular embodiment the present invention relates to a variant alpha-amylase comprising a substitution selected from:

T116N (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116D (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116E (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116Y (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116Q (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

Q,S264K (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V;

### b) G48A+T49I+G107A+H156Y+A181T+N190F+L201F+A209V; or

### c) G48A+T49I+G107A+H156Y+A181T+N190F+L201F+A209V+H68W+K176L+E185P+F201Y+H205Y+K213T+Q360S+D416V+R437W.

In a further particular embodiment the present invention relates to a variant alpha-amylase comprising a substitution selected from:

L196D (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution from:

Q393L (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a combination of substitutions selected from:

T116D+L196D (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity determined by EnzCheck assay after 15 min incubation at 90° C., pH 4.5, 5 ppm Ca²⁺ is at least 45%, at least 50%, particularly at least 55%, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

### T116N;

### T116D;

### T116Q;

### T116E;

### T116Y;

### T181Q;

### T181H;

### Q,S264K;

### Q393L; and

H293Q (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+(Q264S)

### b) G48A+T49I+G107A+H156Y+A181T+N190F+L201F+A209V+(Q264S); or

### c) G48A+T49I+G107A+H156Y+A181T+N190F+L201F+A209V+H68W+K176L+E185P+F201Y+H205Y+K213T+Q360S+D416V+R437W+(Q264S).

In a further particular embodiment the present invention relates to a variant alpha-amylase comprising a substitution selected from:

T116N (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116D (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116Q (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116E (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):
a) H156Y+A181T+N190F+A209V+Q264S;
b) G48A+T49I+G107A+H156Y+A181T+N190F+L201F+A209V+Q264S; or
c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T116Y (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):
a) H156Y+A181T+N190F+A209V+Q264S;

b) G48A+T49I+G107A+H156Y+A181T+N190F+L201F+A209V+Q264S; or
c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T181Q (using SEQ ID NO: 1 for numbering);

wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):
a) H156Y+A181T+N190F+A209V+Q264S;
b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

T181H (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+Q264S;

### b) G48A+T49I+G107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

Q,S264K (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V;

### b) G48A+T49I+G107A+H156Y+A181T+N190F+L201F+A209V; or

### c) G48A+T49I+G107A+H156Y+A181T+N190F+L201F+A209V+H68W+K176L+E185P+F201Y+H205Y+K213T+Q360S+D416V+R437W.

In a further particular embodiment the present invention relates to a variant alpha-amylase comprising a substitution selected from:

Q393L (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+Q264S;

### b) G48A+T49I+G 107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

In a further particular embodiment the present invention relates to a variant alpha-amylase comprising a substitution selected from:

H293Q (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2, and wherein the alpha-amylase variant further comprises a combination of substitutions selected from (using SEQ ID NO: 1 for numbering):

### a) H156Y+A181T+N190F+A209V+Q264S;

### b) G48A+T49I+G 107A+H156Y+A181T+N190F+L201F+A209V+Q264S; or

### c) G48A+T49I+G107A+H156Y+A181T+N190F+L201F+A209V+H68W+K176L+E185P+F201Y+H205Y+K213T+Q360S+D416V+R437W+Q264S.

The variants may further comprise one or more additional alterations, e.g., substitutions, deletions or insertions, at one or more (e.g., several) other positions.

Such further alterations may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

### Parent Alpha-Amylases

In an aspect, the parent has a sequence identity to the mature polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have alpha amylase activity. In one aspect, the amino acid sequence of the parent differs by up to 10 amino acids, e.g., 1, 2, 3, 4, 5, 6, 7, 8, 9, or 10, from the mature polypeptide of SEQ ID NO: 1.

In an aspect, the parent has a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have alpha amylase activity. In one aspect, the amino acid sequence of the parent differs by up to 10 amino acids, e.g., 1, 2, 3, 4, 5, 6, 7, 8, 9, or 10, from the mature polypeptide of SEQ ID NO: 2.

In another aspect, the parent comprises or consists of the amino acid sequence of SEQ ID NO: 1. In another aspect, the parent comprises or consists of the amino acid sequence of SEQ ID NO: 2.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Preparation of Variants

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences. In one embodiment the control sequence(s) is foreign to the polynucleotide.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCI B 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT).

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The prokaryotic host cell may be a Gram-positive bacterium

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Compositions

The present invention also relates to compositions comprising a variant alpha-amylase of the present invention. Preferably the composition also comprises a carrier and/or an excipient. More preferably, the compositions are enriched in such a polypeptide. The term “enriched” indicates that the glucoamylase activity of the composition has been increased, e.g., with an enrichment factor of at least 1.1. Preferably, the compositions are formulated to provide desirable characteristics such as low color, low odor and acceptable storage stability.

The composition may comprise a polypeptide of the present invention as the major enzymatic component, e.g., a mono-component composition. Alternatively, the composition may comprise multiple enzymatic activities.

In a particular embodiment the composition comprises a variant alpha-amylase of the invention and an alpha amylase selected from the group consisting of an alpha-amylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 3. In another particular embodiment the composition comprises a variant alpha-amylase of the invention and an alpha amylase selected from the group consisting of an alpha-amylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 4. In another particular embodiment the composition comprises a variant alpha-amylase of the invention and an alpha amylase selected from the group consisting of an alpha-amylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 5.

In a particular embodiment the composition comprises a variant alpha-amylase of the invention and a pullulanase.

In a particular embodiment the composition further comprises a protease. More particularly in one embodiment the protease is selected from:
a) a Pyrococcus furiosus protease S shown in SEQ ID NO: 6 or a protease having at least 85%, at least 90%, at least 95%, at least 96%, at least 97%, at least 98%, or at least 99%, or 100% sequence identity to the polypeptide of SEQ ID NO: 6;
b) a variant Thermoascus aurantiacus protease, wherein the variant protease comprises one of the following combinations of mutations:

### D79L+S87P+A112P+D142L;

### D79L+S87P+D142L; or

A27K+D79L+Y82F+S87G+D104P+A112P+A126V+D142L; and the protease variant has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 7.

The polypeptide compositions may be prepared in accordance with methods known in the art and may be in the form of a liquid or a dry composition. For instance, the polypeptide composition may be in the form of a granulate or a micro-granulate. The polypeptide to be included in the composition may be stabilized in accordance with methods known in the art.

The compositions may be prepared in accordance with methods known in the art and may be in the form of a liquid or a dry composition. For instance, the composition may be in the form of granulate or microgranulate. The variant may be stabilized in accordance with methods known in the art.

The compositions may be prepared in accordance with methods known in the art and may be in the form of a liquid or a dry composition. The compositions may be stabilized in accordance with methods known in the art.

The enzyme composition of the present invention may be in any form suitable for use, such as, for example, a crude fermentation broth with or without cells removed, a cell lysate with or without cellular debris, a semi-purified or purified enzyme composition, or a host cell, as a source of the enzymes.

The enzyme composition may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

Examples are given below of preferred uses of the polypeptide or polypeptide compositions of the invention. The dosage of the polypeptide composition of the invention and other conditions under which the composition is used may be determined on the basis of methods known in the art.

The above compositions are suitable for use in liquefaction, saccharification, and/or fermentation processes, preferably in starch conversion, especially for producing syrup and fermentation products, such as ethanol.

Examples are given below of preferred uses of the polypeptide compositions of the present invention. The dosage of the polypeptide composition of the invention and other conditions under which the composition is used may be determined on the basis of methods known in the art.

### Uses

The alpha-amylase variants according to the invention are particularly useful in starch processing, more particularly in liquefaction of starch containing material.

### Starch Processing

Native starch consists of microscopic granules, which are insoluble in water at room temperature. When aqueous starch slurry is heated, the granules swell and eventually burst, dispersing the starch molecules into the solution. At temperatures up to about 50° C. to 75° C. the swelling may be reversible. However, with higher temperatures an irreversible swelling called “gelatinization” begins. During this “gelatinization” process there is a dramatic increase in viscosity. Granular starch to be processed may be a highly refined starch quality, preferably at least 90%, at least 95%, at least 97% or at least 99.5% pure or it may be a more crude starch-containing materials comprising (e.g., milled) whole grains including non-starch fractions such as germ residues and fibers. The raw material, such as whole grains, may be reduced in particle size, e.g., by milling, in order to open up the structure and allowing for further processing. In dry milling whole kernels are milled and used. Wet milling gives a good separation of germ and meal (starch granules and protein) and is often applied at locations where the starch hydrolysate is used in the production of, e.g., syrups. Both dry and wet milling is well known in the art of starch processing and may be used in a process of the invention. Methods for reducing the particle size of the starch containing material are well known to those skilled in the art.

As the solids level is 30-40% in a typical industrial process, the starch has to be thinned or “liquefied” so that it can be suitably processed. This reduction in viscosity is primarily attained by enzymatic degradation in current commercial practice.

Liquefaction is carried out in the presence of an alpha-amylase, preferably a bacterial alpha-amylase and/or acid fungal alpha-amylase. In an embodiment, a phytase is also present during liquefaction. In an embodiment, viscosity reducing enzymes such as a xylanase and/or beta-glucanase is also present during liquefaction.

During liquefaction, the long-chained starch is degraded into branched and linear shorter units (maltodextrins) by an alpha-amylase. Liquefaction may be carried out as a three-step hot slurry process. The slurry is heated to between 60-95° C. (e.g., 70-90° C., such as 77-86° C., 80-85° C., 83-85° C.) and an alpha-amylase is added to initiate liquefaction (thinning).

The slurry may in an embodiment be jet-cooked at between 95-140° C., e.g., 105-125° C., for about 1-15 minutes, e.g., about 3-10 minutes, especially around 5 minutes. The slurry is then cooled to 60-95° C. and more alpha-amylase is added to obtain final hydrolysis (secondary liquefaction). The jet-cooking process is carried out at pH 4.5-6.5, typically at a pH between 5 and 6. The alpha-amylase may be added as a single dose, e.g., before jet cooking.

The liquefaction process is carried out at between 70-95° C., such as 80-90° C., such as around 85° C., for about 10 minutes to 5 hours, typically for 1-2 hours. The pH is between 4 and 7, such as between 4.5 and 5.5. In order to ensure optimal enzyme stability under these conditions, calcium may optionally be added (to provide 1-60 ppm free calcium ions, such as about 40 ppm free calcium ions). After such treatment, the liquefied starch will typically have a “dextrose equivalent” (DE) of 10-15.

Generally liquefaction and liquefaction conditions are well known in the art.

Saccharification may be carried out using conditions well-known in the art with a carbohydrate-source generating enzyme, in particular a glucoamylase, or a beta-amylase and optionally a debranching enzyme, such as an isoamylase or a pullulanase. For instance, a full saccharification step may last from about 24 to about 72 hours. However, it is common to do a pre-saccharification of typically 40-90 minutes at a temperature between 30-65° C., typically about 60° C., followed by complete saccharification during fermentation in a simultaneous saccharification and fermentation (SSF) process. Saccharification is typically carried out at a temperature in the range of 20-75° C., e.g., 25-65° C. and 40-70° C., typically around 60° C., and at a pH between about 4 and 5, normally at about pH 4.5.

The saccharification and fermentation steps may be carried out either sequentially or simultaneously. In an embodiment, saccharification and fermentation are performed simultaneously (referred to as “SSF”). However, it is common to perform a pre-saccharification step for about 30 minutes to 2 hours (e.g., 30 to 90 minutes) at a temperature of 30 to 65° C., typically around 60° C. which is followed by a complete saccharification during fermentation referred to as simultaneous saccharification and fermentation (SSF). The pH is usually between 4.2-4.8, e.g., pH 4.5. In a simultaneous saccharification and fermentation (SSF) process, there is no holding stage for saccharification, rather, the yeast and enzymes are added together. In a typical saccharification process, maltodextrins produced during liquefaction are converted into dextrose by adding a glucoamylase and a debranching enzyme, such as an isoamylase (U.S. Pat. No. 4,335,208) or a pullulanase. The temperature is lowered to 60° C., prior to the addition of the glucoamylase and debranching enzyme. The saccharification process proceeds for 24-72 hours. Prior to addition of the saccharifying enzymes, the pH is reduced to below 4.5, while maintaining a high temperature (above 95° C.), to inactivate the liquefying alpha-amylase. This process reduces the formation of short oligosaccharide called “panose precursors,” which cannot be hydrolyzed properly by the debranching enzyme. Normally, about 0.2-0.5% of the saccharification product is the branched trisaccharide panose (Glc pα1-6Glc pα1-4Glc), which cannot be degraded by a pullulanase. If active amylase from the liquefaction remains present during saccharification (i.e., no denaturing), the amount of panose can be as high as 1-2%, which is highly undesirable since it lowers the saccharification yield significantly.

Other fermentation products may be fermented at conditions and temperatures well known to persons skilled in the art, suitable for the fermenting organism in question.

The fermentation product may be recovered by methods well known in the art, e.g., by distillation. Examples of carbohydrate-source generating enzymes are disclosed in the “Enzymes” section below.

In a particular embodiment, the process of the invention further comprises, prior to the conversion of a starch-containing material to sugars/dextrins the steps of:

(x) reducing the particle size of the starch-containing material; and
(y) forming a slurry comprising the starch-containing material and water.

In an embodiment, the starch-containing material is milled to reduce the particle size. In an embodiment the particle size is reduced to between 0.05-3.0 mm, preferably 0.1-0.5 mm, or so that at least 30%, preferably at least 50%, more preferably at least 70%, even more preferably at least 90% of the starch-containing material fits through a sieve with a 0.05-3.0 mm screen, preferably 0.1-0.5 mm screen.

The aqueous slurry may contain from 10-55 wt. % dry solids (DS), preferably 25-45 wt. % dry solids (DS), more preferably 30-40 wt. % dry solids (DS) of starch-containing material. Conventional starch-conversion processes, such as liquefaction and saccharification processes are described, e.g., in U.S. Pat. No. 3,912,590, EP 252730 and EP 063909, which are incorporated herein by reference.

In an embodiment, the conversion process degrading starch to lower molecular weight carbohydrate components such as sugars or fat replacers includes a debranching step. In the case of converting starch into a sugar, the starch is depolymerized. Such a depolymerization process consists of, e.g., a pre-treatment step and two or three consecutive process steps, i.e., a liquefaction process, a saccharification process, and depending on the desired end-product, an optional isomerization process.

When the desired final sugar product is, e.g., high fructose syrup the dextrose syrup may be converted into fructose. After the saccharification process, the pH is increased to a value in the range of 6-8, e.g., pH 7.5, and the calcium is removed by ion exchange. The dextrose syrup is then converted into high fructose syrup using, e.g., an immobilized glucose isomerase.

### Production of Fermentation Products

Fermentable sugars (e.g., dextrins, monosaccharides, particularly glucose) are produced from enzymatic saccharification. These fermentable sugars may be further purified and/or converted to useful sugar products. In addition, the sugars may be used as a fermentation feedstock in a microbial fermentation process for producing end-products, such as alcohol (e.g., ethanol, and butanol), organic acids (e.g., succinic acid, 3-HP and lactic acid), sugar alcohols (e.g., glycerol), ascorbic acid intermediates (e.g., gluconate, 2-keto-D-gluconate, 2,5-diketo-D-gluconate, and 2-keto-L-gulonic acid), amino acids (e.g., lysine), proteins (e.g., antibodies and fragment thereof).

In an embodiment, the fermentable sugars obtained during the liquefaction process steps are used to produce alcohol and particularly ethanol. In ethanol production, an SSF process is commonly used wherein the saccharifying enzymes and fermenting organisms (e.g., yeast) are added together and then carried out at a temperature of 30-40° C.

The organism used in fermentation will depend on the desired end-product. Typically, if ethanol is the desired end product yeast will be used as the fermenting organism. In some preferred embodiments, the ethanol-producing microorganism is a yeast and specifically Saccharomyces such as strains of S. cerevisiae (U.S. Pat. No. 4,316,956). A variety of S. cerevisiae are commercially available and these include but are not limited to FALI (Fleischmann's Yeast), SUPERSTART (Alltech), FERMIOL (DSM Specialties), RED STAR (Lesaffre) and Angel alcohol yeast (Angel Yeast Company, China). The amount of starter yeast employed in the methods is an amount effective to produce a commercially significant amount of ethanol in a suitable amount of time, (e.g., to produce at least 10% ethanol from a substrate having between 25-40% DS in less than 72 hours). Yeast cells are generally supplied in amounts of about 10⁴ to about 10¹², and preferably from about 10⁷ to about 10¹⁰ viable yeast count per mL of fermentation broth. After yeast is added to the mash, it is typically subjected to fermentation for about 24-96 hours, e.g., 35-60 hours. The temperature is between about 26-34° C., typically at about 32° C., and the pH is from pH 3-6, e.g., around pH 4-5.

The fermentation may include, in addition to a fermenting microorganisms (e.g., yeast), nutrients, and additional enzymes, including phytases. The use of yeast in fermentation is well known in the art.

In further embodiments, use of appropriate fermenting microorganisms, as is known in the art, can result in fermentation end product including, e.g., glycerol, 1,3-propanediol, gluconate, 2-keto-D-gluconate, 2,5-diketo-D-gluconate, 2-keto-L-gulonic acid, succinic acid, lactic acid, amino acids, and derivatives thereof. More specifically when lactic acid is the desired end product, a Lactobacillus sp. (L. casei) may be used; when glycerol or 1,3-propanediol are the desired end-products E. coli may be used; and when 2-keto-D-gluconate, 2,5-diketo-D-gluconate, and 2-keto-L-gulonic acid are the desired end products, Pantoea citrea may be used as the fermenting microorganism. The above enumerated list are only examples and one skilled in the art will be aware of a number of fermenting microorganisms that may be used to obtain a desired end product.

Processes for Producing Fermentation Products from Gelatinized Starch-Containing Material

In this aspect, the invention relates to processes for producing fermentation products, especially ethanol, from starch-containing material, which process includes a liquefaction step and sequentially or simultaneously performed saccharification and fermentation steps. Consequently, the invention relates to a process for producing a fermentation product from starch-containing material comprising the steps of:

(a) liquefying starch-containing material in the presence of an alpha-amylase variant of the invention;

(b) saccharifying the liquefied material obtained in step (a) using a carbohydrate-source generating enzyme;

(c) fermenting using a fermenting organism.

The fermentation product, such as especially ethanol, may optionally be recovered after fermentation, e.g., by distillation. The fermenting organism is preferably yeast, preferably a strain of Saccharomyces cerevisiae. In a particular embodiment, the process of the invention further comprises, prior to step (a), the steps of:

x) reducing the particle size of the starch-containing material, preferably by milling (e.g., using a hammer mill);
y) forming a slurry comprising the starch-containing material and water.
In an embodiment, the particle size is smaller than a #7 screen, e.g., a #6 screen. A #7 screen is usually used in conventional prior art processes. The aqueous slurry may contain from 10-55, e.g., 25-45 and 30-40, w/w % dry solids (DS) of starch-containing material. The slurry is heated to above the gelatinization temperature and an alpha-amylase variant may be added to initiate liquefaction (thinning). The slurry may in an embodiment be jet-cooked to further gelatinize the slurry before being subjected to alpha-amylase in step (a). Liquefaction may in an embodiment be carried out as a three-step hot slurry process. The slurry is heated to between 60-95° C., preferably between 70-90° C., such as preferably between 80-85° C. at pH 4-6, preferably 4.5-5.5, and alpha-amylase variant, optionally together with a pullulanase and/or protease, preferably metalloprotease, are added to initiate liquefaction (thinning). In an embodiment the slurry may then be jet-cooked at a temperature between 95-140° C., preferably 100-135° C., such as 105-125° C., for about 1-15 minutes, preferably for about 3-10 minutes, especially around about 5 minutes. The slurry is cooled to 60-95° C. and more alpha-amylase variant and optionally pullulanase variant and/or protease, preferably metalloprotease, is(are) added to finalize hydrolysis (secondary liquefaction). The liquefaction process is usually carried out at pH 4.0-6, in particular at a pH from 4.5 to 5.5. Saccharification step (b) may be carried out using conditions well known in the art. For instance, a full saccharification process may last up to from about 24 to about 72 hours, however, it is common only to do a pre-saccharification of typically 40-90 minutes at a temperature between 30-65° C., typically about 60° C., followed by complete saccharification during fermentation in a simultaneous saccharification and fermentation process (SSF process). Saccharification is typically carried out at temperatures from 20-75° C., preferably from 40-70° C., typically around 60° C., and at a pH between 4 and 5, normally at about pH 4.5. The most widely used process to produce a fermentation product, especially ethanol, is a simultaneous saccharification and fermentation (SSF) process, in which there is no holding stage for the saccharification, meaning that a fermenting organism, such as yeast, and enzyme(s), may be added together. SSF may typically be carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours.
Glucoamylase Present and/or Added in Saccharification and/or Fermentation

The carbohydrate-source generating enzyme present during saccharification may in one embodiment be a glucoamylase. A glucoamylase is present and/or added in saccharification in a process of the invention.

In an embodiment the glucoamylase present and/or added in saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably T. emersonii (e.g., as disclosed in WO99/28448) or a strain of Trametes, preferably T. cingulata (e.g., as disclosed in WO06/069289), or a strain of Pycnoporus, preferably P. sanguineus (e.g., as disclosed in WO11/066576), or a strain of Gloeophyllum, such as G. serpiarium or G. trabeum (e.g., as disclosed in WO11/068803), or a strain of the Nigrofomes (e.g., WO12/064351).

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

According to a preferred embodiment of the invention the glucoamylase is present and/or added in saccharification and/or fermentation in combination with an alpha-amylase. Examples of suitable alpha-amylase are described below.

Alpha-Amylase Present and/or Added in Saccharification and/or Fermentation

In an embodiment an alpha-amylase is present and/or added in saccharification and/or fermentation in the processes of the invention. In a preferred embodiment the alpha-amylase is of fungal or bacterial origin. In a preferred embodiment the alpha-amylase is a fungal acid stable alpha-amylase. A fungal acid stable alpha-amylase is an alpha-amylase that has activity in the pH range of 3.0 to 7.0 and preferably in the pH range from 3.5 to 6.5, including activity at a pH of about 4.0, 4.5, 5.0, 5.5, and 6.0.

In a preferred embodiment the alpha-amylase present and/or added in saccharification and/or fermentation is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-binding domain, such as the one shown in SEQ ID NO: 8 herein, or a variant thereof.

In an embodiment the alpha-amylase is derived from a Rhizomucor pusillus with an Aspergillus niger glucoamylase linker and starch-binding domain (SBD), preferably disclosed as SEQ ID NO: 8 herein, preferably having one or more of the following substitutions: G128D, D143N, preferably G128D+D143N (using SEQ ID NO: 8 for numbering), and wherein the alpha-amylase variant present and/or added in saccharification and/or fermentation has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 8 herein.

In a preferred embodiment the ratio between glucoamylase and alpha-amylase present and/or added during saccharification and/or fermentation may preferably be in the range from 500:1 to 1:1, such as from 250:1 to 1:1, such as from 100:1 to 1: 1, such as from 100: 2 to 100:50, such as from 100:3 to 100:70.

### Starch-Containing Materials

Any suitable starch-containing starting material may be used in a process of the present invention. The starting material is generally selected based on the desired fermentation product. Examples of starch-containing starting materials, suitable for use in the processes of the present invention, include barley, beans, cassava, cereals, corn, milo, peas, potatoes, rice, rye, sago, sorghum, sweet potatoes, tapioca, wheat, and whole grains, or any mixture thereof. The starch-containing material may also be a waxy or non-waxy type of corn and barley. In a preferred embodiment the starch-containing material is corn. In a preferred embodiment the starch-containing material is wheat.

### Fermentation Products

The term “fermentation product” means a product produced by a method or process including fermenting using a fermenting organism. Fermentation products include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. In an preferred embodiment the fermentation product is ethanol.

Starch Slurry Processing with Stillage

Milled starch-containing material is combined with water and recycled thin-stillage resulting in an aqueous slurry. The slurry can comprise between 15 to 55% ds w/w (e.g., 20 to 50%, 25 to 50%, 25 to 45%, 25 to 40%, 20 to 35% and 30-36% ds). In some embodiments, the recycled thin-stillage (backset) is in the range of about 10 to 70% v/v (e.g., 10 to 60%, 10 to 50%, 10 to 40%, 10 to 30%, 10 to 20%, 20 to 60%, 20 to 50%, 20 to 40% and also 20 to 30%). Once the milled starch-containing material is combined with water and backset, the pH is not adjusted in the slurry. Further the pH is not adjusted after the addition of a phytase and optionally an alpha-amylase to the slurry. In an embodiment, the pH of the slurry will be in the range of about pH 4.5 to less than about 6.0 (e.g., pH 4.5 to 5.8, pH 4.5 to 5.6, pH 4.8 to 5.8, pH 5.0 to 5.8, pH 5.0 to 5.4, pH 5.2 to 5.5 and pH 5.2 to 5.9). The pH of the slurry may be between about pH 4.5 and 5.2 depending on the amount of thin stillage added to the slurry and the type of material comprising the thin stillage. For example, the pH of the thin stillage may be between pH 3.8 and pH 4.5.

During ethanol production, acids can be added to lower the pH in the beer well, to reduce the risk of microbial contamination prior to distillation.

In some embodiments, a phytase is added to the slurry. In other embodiments, in addition to phytase, an alpha-amylase is added to the slurry. In some embodiments, a phytase and alpha-amylase are added to the slurry sequentially. In other embodiments, a phytase and alpha-amylase are added simultaneously. In some embodiments, the slurry comprising a phytase and optionally, an alpha-amylase, are incubated (pretreated) for a period of about 5 minutes to about 8 hours (e.g., 5 minutes to 6 hours, 5 minutes to 4 hours, 5 minutes to 2 hours, and 15 minutes to 4 hours). In other embodiments, the slurry is incubated at a temperature in the range of about 40 to 115° C. (e.g., 45 to 80° C., 50 to 70° C., 50 to 75° C., 60 to 110° C., 60 to 95° C., 70 to 110° C., 70 to 85° C. and 77 to 86° C.).

In other embodiments, the slurry is incubated at a temperature of about 0 to about 30° C. (e.g., 0 to 25° C., 0 to 20° C., 0 to 15° C., 0 to 10° C. and 0 to 5° C.) below the starch gelatinization temperature of the starch-containing material. In some embodiments, the temperature is below about 68° C., below about 65° C., below about 62° C., below about 60° C. and below about 55° C. In some embodiments, the temperature is above about 45° C., above about 50° C., above about 55° C. and above about 60° C. In some embodiments, the incubation of the slurry comprising a phytase and an alpha-amylase at a temperature below the starch gelatinization temperature is referred to as a primary (1°) liquefaction.

In one embodiment, the milled starch-containing material is corn or milo. The slurry comprises 25 to 40% DS, the pH is in the range of 4.8 to 5.2, and the slurry is incubated with a phytase and optionally an alpha-amylase for 5 minutes to 2 hours, at a temperature range of 60 to 75° C.

In a further liquefaction step, the incubated or pretreated starch-containing material is exposed to an increase in temperature such as about 0 to about 45° C. above the starch gelatinization temperature of the starch-containing material (e.g., 70° C. to 120° C., 70° C. to 110° C., and 70° C. to 90° C.) for a period of time of about 2 minutes to about 6 hours (e.g., 2 minutes to 4 hours, 90 minutes, 140 minutes and 90 to 140 minutes) at a pH of about 4.0 to 5.5 more preferably between 1 hour to 2 hours. The temperature can be increased by a conventional high temperature jet cooking system for a short period of time, for example, for 1 to 15 minutes. Then the starch maybe further hydrolyzed at a temperature ranging from about 75° C. to 95° C. (e.g., 80° C. to 90° C. and 80° C. to 85° C.) fora period of about 15 to 150 minutes (e.g., 30 to 120 minutes). In a preferred embodiment, the pH is not adjusted during these process steps and the pH of the liquefied mash is in the range of about pH 4.0 to pH 5.8 (e.g., pH 4.5 to 5.8, pH 4.8 to 5.4, and pH 5.0 to 5.2). In some embodiments, a second dose of thermostable alpha-amylase is added to the secondary liquefaction step, but in other embodiments there is no additional dosage of alpha-amylase.

The incubation and liquefaction steps may be followed by saccharification and fermentation steps well known in the art.

### Distillation

Optionally, following fermentation, an alcohol (e.g., ethanol) may be extracted by, for example, distillation and optionally followed by one or more process steps.

In some embodiments, the yield of ethanol produced by the methods provided herein is at least 8%, at least 10%, at least 12%, at least 14%, at least 15%, at least 16%, at least 17% and at least 18% (v/v) and at least 23% v/v. The ethanol obtained according to the process provided herein may be used as, for example, fuel ethanol, drinking ethanol, i.e., potable neutral spirits, or industrial ethanol.

### By-Products

Left over from the fermentation is the grain, which is typically used for animal feed either in liquid or dried form. In further embodiments, the end product may include the fermentation co-products such as distiller's dried grains (DDG) and distiller's dried grain plus solubles (DDGS), which may be used, for example, as an animal feed.

Further details on how to carry out liquefaction, saccharification, fermentation, distillation, and recovery of ethanol are well known to the skilled person.

According to the process provided herein, the saccharification and fermentation may be carried out simultaneously or separately.

### Fermenting Organisms

The term “fermenting organism” refers to any organism, including bacterial and fungal organisms, such as yeast and filamentous fungi, suitable for producing a desired fermentation product. Suitable fermenting organisms are able to ferment, i.e., convert, fermentable sugars, such as arabinose, fructose, glucose, maltose, mannose, or xylose, directly or indirectly into the desired fermentation product.

Examples of fermenting organisms include fungal organisms such as yeast. Preferred yeast include strains of Saccharomyces, in particular Saccharomyces cerevisiae or Saccharomyces uvarum; strains of Pichia, in particular Pichia stipitis such as Pichia stipitis CBS 5773 or Pichia pastoris; strains of Candida, in particular Candida arabinofermentans, Candida boidinii, Candida diddensii, Candida shehatae, Candida sonorensis, Candida tropicalis, or Candida utilis. Other fermenting organisms include strains of Hansenula, in particular Hansenula anomala or Hansenula polymorpha; strains of Kluyveromyces, in particular Kluyveromyces fragilis or Kluyveromyces marxianus; and strains of Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes, and strains of Thermoanaerobacter, in particular Thermoanaerobacter BG1L1 (Appl. Microbiol. Biotech. 77: 61-86), Thermoanarobacter ethanolicus, Thermoanaerobacter mathranii, or Thermoanaerobacter thermosaccharolyticum. Strains of Lactobacillus are also envisioned as are strains of Corynebacterium glutamicum R, Bacillus the thermoglucosidaisus, and Geobacillus thermoglucosidasius.

In an embodiment, the fermenting organism is a C6 sugar fermenting organism, such as a strain of, e.g., Saccharomyces cerevisiae.

In an embodiment, the fermenting organism is a C5 sugar fermenting organism, such as a strain of, e.g., Saccharomyces cerevisiae.

In one embodiment, the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Yeast is the preferred fermenting organism for ethanol fermentation. Preferred are strains of Saccharomyces, especially strains of the species Saccharomyces cerevisiae, preferably strains which are resistant towards high levels of ethanol, i.e., up to, e.g., about 10, 12, 15 or 20 vol. % or more ethanol.

In an embodiment, the C5 utilizing yeast is a Saccharomyces cerevisea strain disclosed in WO 2004/085627.

In an embodiment, the fermenting organism is a C5 eukaryotic microbial cell concerned in WO 2010/074577 (Nedalco).

In an embodiment, the fermenting organism is a transformed C5 eukaryotic cell capable of directly isomerize xylose to xylulose disclosed in US 2008/0014620.

In an embodiment, the fermenting organism is a C5 sugar fermentating cell disclosed in WO 2009/109633.

Commercially available yeast include LNF SA-1, LNF BG-1, LNF PE-2, and LNF CAT-1 (available from LNF Brazil), RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

The fermenting organism capable of producing a desired fermentation product from fermentable sugars is preferably grown under precise conditions at a particular growth rate. When the fermenting organism is introduced into/added to the fermentation medium the inoculated fermenting organism pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism enters “stationary phase”. After a further period of time the fermenting organism enters the “death phase” where the number of viable cells declines.

### Fermentation

The fermentation conditions are determined based on, e.g., the kind of plant material, the available fermentable sugars, the fermenting organism(s) and/or the desired fermentation product. One skilled in the art can easily determine suitable fermentation conditions. The fermentation may be carried out at conventionally used conditions. Preferred fermentation processes are anaerobic processes.

For example, fermentations may be carried out at temperatures as high as 75° C., e.g., between 40-70° C., such as between 50-60° C. However, bacteria with a significantly lower temperature optimum down to around room temperature (around 20° C.) are also known. Examples of suitable fermenting organisms can be found in the “Fermenting Organisms” section above.

For ethanol production using yeast, the fermentation may go on for 24 to 96 hours, in particular for 35 to 60 hours. In an embodiment the fermentation is carried out at a temperature between 20 to 40° C., preferably 26 to 34° C., in particular around 32° C. In an embodiment the pH is from pH 3 to 6, preferably around pH 4 to 5.

Other fermentation products may be fermented at temperatures known to the skilled person in the art to be suitable for the fermenting organism in question. Fermentation is typically carried out at a pH in the range between 3 and 7, preferably from pH 3.5 to 6, such as around pH 5. Fermentations are typically ongoing for 6-96 hours.

The processes of the invention may be performed as a batch or as a continuous process. Fermentations may be conducted in an ultrafiltration system wherein the retentate is held under recirculation in the presence of solids, water, and the fermenting organism, and wherein the permeate is the desired fermentation product containing liquid. Equally contemplated are methods/processes conducted in continuous membrane reactors with ultrafiltration membranes and where the retentate is held under recirculation in presence of solids, water, and the fermenting organism(s) and where the permeate is the fermentation product containing liquid.

After fermentation the fermenting organism may be separated from the fermented slurry and recycled.

### Fermentation Medium

The phrase “fermentation media” or “fermentation medium” refers to the environment in which fermentation is carried out and comprises the fermentation substrate, that is, the carbohydrate source that is metabolized by the fermenting organism(s).

The fermentation medium may comprise other nutrients and growth stimulator(s) for the fermenting organism(s). Nutrient and growth stimulators are widely used in the art of fermentation and include nitrogen sources, such as ammonia; vitamins and minerals, or combinations thereof.

### Recovery

Subsequent to fermentation, the fermentation product may be separated from the fermentation medium. The fermentation medium may be distilled to extract the desired fermentation product or the desired fermentation product may be extracted from the fermentation medium by micro or membrane filtration techniques. Alternatively, the fermentation product may be recovered by stripping. Methods for recovery are well known in the art.

The invention is further defined in the following numbered paragraphs:

1. An alpha-amylase variant comprising a substitution at a position corresponding to position 116, 393, 181, 293, 264, or 196 of SEQ ID NO: 1, in particular one or more substitutions selected from the group consisting of T116N, T116D, T116E, T116Q, T116Y, T181H, T181Q, S264K, L196D, H293Q, and Q393L, wherein the variant has at least 75%, at least 80%, at least 85%, at least 90%, at least 95%, at least 96%, at least 97%, at least 98%, or at least 99%, but less than 100% sequence identity to amino acids 1 to 483 of SEQ ID NO: 1, amino acids 1 to 481 of SEQ ID NO: 2; and wherein the variant has alpha-amylase activity.

2. The alpha-amylase variants of paragraph 1, comprising a substitution at a position corresponding to position 116, 393, 264, or 196 of SEQ ID NO: 1, in particular one or more substitutions selected from the group consisting of T116N, T116D, T116Q, T116Y, S264K, L196D, Q393L, and T116D+L196D; wherein the variants have an increased stability compared to a parent alpha-amylase selected from the group consisting of amino acids 1 to 483 of SEQ ID NO: 1, or amino acids 1 to 481 of SEQ ID NO: 2, and wherein the increased stability is measured as residual alpha-amylase activity determined by EnzCheck assay after 15 min incubation at 90° C., pH 4.5, 5 ppm Ca2+.

3. The alpha-amylase variants according to paragraph 2, comprising a substitution or combination of substitutions selected from:

### T116N;

### T116D;

### T116Q;

### T116E;

### T116Y;

### S264K;

### L196D;

### Q393L; and

T116D+L196D (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and the residual alpha-amylase activity is at least 45%, at least 50%, particularly at least 55%.

4. The alpha-amylase variants of paragraph 1, comprising a substitution at a position corresponding to position 116, 181, 393, 264, or 293 of SEQ ID NO: 1, in particular one or more substitutions selected from the group consisting of T116N, T116D, T116Q, T116E, T116Y, T181H, T181Q, S264K, H293Q, and Q393L, wherein the variants are capable of generating a liquefact having a dextrose equivalent (DE) value higher than the DE value generated by a parent alpha-amylase selected from the group consisting of amino acids 1 to 483 of SEQ ID NO: 1, or amino acids 1 to 481 of SEQ ID NO: 2.

5. The alpha-amylase variant of paragraph 4, comprising a substitution or combination of substitutions selected from:

### T116N;

### T116D;

### T116Q;

### T116E;

### T116Y;

### T181Q;

### T181H;

### S264K;

### Q393L; and

H293Q (using SEQ ID NO: 1 for numbering);
wherein the variants have at least 75%, at least 80%, at least 85%, at least 90%, at least 95%, at least 96%, at least 97%, at least 98%, or at least 99%, but less than 100% sequence identity to amino acids 1 to 481 of SEQ ID NO: 2, and wherein the DE value is at least 2 higher than the DE value generated by the parent alpha amylase of SEQ ID NO: 2.

6. The alpha-amylase variants of paragraph 5, wherein the variants are capable of generating a liquefact having a dextrose equivalent (DE) value is in the range from 12.5 to 22, particularly from 13 to 21.

7. The variant of paragraph 1, wherein the variant alpha-amylase is isolated.

8. The variant of any of paragraphs 1-7, wherein the number of substitutions is 1-20, e.g., 1-10 and 1-5, such as 1, 2, 3, 4, 5, 6, 7, 8, 9 or 10 alterations.

9. An polynucleotide encoding the variant of any of paragraphs 1-8.

10. A nucleic acid construct comprising the polynucleotide of paragraph 9.

11. An expression vector comprising the polynucleotide of paragraph 9.

12. A host cell comprising the polynucleotide of paragraph 9.

13. A composition comprising the variant alpha-amylase of any of the paragraphs 1-8.

14. The composition according to paragraph 13, further comprising an alpha-amylase selected from the group consisting of:

a) an alpha-amylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 3;
b) an alpha-amylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 4;
c) an alpha-amylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 5.

15. The composition according to any of paragraphs 13-14, further comprising a protease.

16. The composition according to any of the paragraphs 13-15, wherein the protease is selected from:

a) a Pyrococcus furiosus protease S shown in SEQ ID NO: 6 or a protease having at least 85%, at least 90%, at least 95%, at least 96%, at least 97%, at least 98%, or at least 99%, or 100% sequence identity to the polypeptide of SEQ ID NO: 6;
b) a variant Thermoascus aurantiacus protease, wherein the variant protease comprises one of the following combinations of mutations:

### D79L+S87P+A112P+D142L;

### D79L+S87P+D142L; or

A27K+D79L+Y82F+S87G+D104P+A112P+A126V+D142L; and the protease variant has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 7.

17. A use of the variant of any of paragraphs 1-8 or the composition according to any of paragraphs 13-16 for liquefying a starch-containing material.

18. A process for producing a syrup from starch-containing material comprising the steps of:

a) liquefying the starch-containing material at a temperature above the initial gelatinization temperature in the presence of a variant alpha-amylase according to any of the paragraphs 1-8 or a composition of any of paragraphs 13-16; and
b) saccharifying the produce of step a) in the presence of a glucoamylase.

19. The process according to paragraph 18, wherein step b) is performed in the presence of a glucoamylase and:

i) a fungal alpha-amylase;
ii) an isoamylase;
iii) a fungal alpha-amylase and an isoamylase.

20. The process according to any of paragraphs 18-19, wherein a pullulanase is present in step b).

21. The process according to any of the paragraphs 18-20 further comprising:

c) fermenting the product of step b) using a fermenting organism to produce a fermentation product.

22. The process of paragraph 21, wherein the fermenting organism is a yeast and the fermentation product is alcohol.

23. The process of paragraph 22, wherein the yeast is Saccharomyces cerevisiae and the alcohol is ethanol.

24. The process of any of the paragraphs 18-23, wherein steps b) and c) are performed simultaneously.

25. A method of producing an alpha-amylase variant, comprising cultivating the host cell of paragraph 12 under conditions suitable for expression of the variant; and optionally recovering the variant.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

### EXAMPLES

### Assays:

### Assay for Determination of Residual Alpha-Amylase Activity

For the determination of residual amylase activity an EnzChek® Ultra Amylase Assay Kit (E33651, Invitrogen, La Jolla, Calif., USA) was used.

The substrate is a corn starch derivative, DQ™ starch, which is corn starch labeled with BODIPY® FL dye to such a degree that fluorescence is quenched. One vial containing approx. 1 mg lyophilized substrate is dissolved in 100 microliters of 50 mM sodium acetate (pH 4.0). The vial is vortexed for 20 seconds and left at room temperature, in the dark, with occasional mixing until dissolved. Then 900 microliters of 100 mM potassium acetate, 0.01% (w/v) TRITON® X100, 0.125 mM CaCl₂, pH 5.5 is added, vortexed thoroughly and stored at room temperature, in the dark until use. The stock substrate working solution is prepared by diluting 10-fold in residual activity buffer (100 mM potassium acetate, 0.01% (w/v) TRITON® X100, 0.125 mM CaCl₂, pH 5.5). Immediately after incubation, the enzyme is diluted to a concentration of 10 ng enzyme protein/mL in 100 mM potassium acetate, 0.01% (w/v) TRITON® X100, 0.125 mM CaCl₂, pH 5.5.

For the assay, 25 microliters of the substrate working solution is mixed for 10 second with 25 microliters of the diluted enzyme in a black 384-well microtiter plate. The fluorescence intensity is measured (excitation: 485 nm, emission: 555 nm) every 30 second for 10 minutes in each well at 25° C. and the Vₘₐₓ is calculated as the slope of the plot of fluorescence intensity against time and residual activity is determined by normalization to control samples for each setup. The plot should be linear and the residual activity assay has been adjusted so that the diluted reference enzyme solution is within the linear range of the activity assay.

### Example 1: Thermostability of Alpha-Amylase Variants at pH 4.5

The thermostability of a reference alpha-amylase and alpha-amylase variants thereof were determined by incubating the reference alpha-amylase and variants at pH 4.5 and temperature of 90° C. with 0.125 mM CaCl₂) for 15 min followed by residual activity determination using the EnzChek® substrate (EnzChek® Ultra Amylase assay kit, E33651, Molecular Probes). Purified enzyme samples were diluted to working concentrations of 10 ppm (micrograms/ml) in enzyme dilution buffer (10 mM potassium acetate, 0.01% (w/v) TRITON® X100, 0.125 mM CaCl₂, pH 4.5). Twenty microliters enzyme sample was transferred to 96-well PCR microtiter plate and 180 microliters stability buffer (100 mM potassium acetate, 0.01% (w/v) TRITON® X100, 0.125 mM CaCl₂, pH 4.5) was added to each well and mixed. Before incubation at 90° C., 20 microliters was withdrawn and stored on ice as control samples. Incubation was performed in a PCR machine for 15 minutes at 90° C.

After incubation samples were diluted to 10 ng/ml in residual activity buffer (100 mM potassium acetate, 0.01% (w/v) TRITON® X100, 0.125 mM CaCl₂, pH 5.5) and 25 microliters diluted enzyme was transferred to a black 384-well microtiter plate. Residual activity was determined using the EnzChek® substrate by adding 25 microliters substrate solution (100 micrograms/ml) to each well. Fluorescence was determined at 25° C. every 30 second for 10 minutes using excitation at 485 nm and emission at 555 nm (fluorescence reader SpectraMax M2e, Molecular Devices). The residual activity was normalized to control samples for each setup. Using this assay setup the residual activity was determined as a measure of thermostability for the reference alpha-amylase and variants thereof as shown in Tables 1.

TABLE 1
Conditions: pH 4.5, 0.125 mM CaCl₂, 90° C. for 15 minutes

| Substitution | Residual activity (%) |
|---|---|
| Reference | 41 |
| Q393L | 63 |
| T116Q | 63 |
| T116N | 58 |
| S264K (Q264K) | 51 |
| T116D L196D | 68 |

### Example 2: Use of L-Type Alpha-Amylase in Starch Liquefaction

The variant amylases of the invention were tested for use in a 4.3 pH, 30 ppm Na, 5 ppm Ca starch liquefaction process.

Substrate Preparation:

Starch slurry was prepared from powdered bag starch of an 88% dry solids and DI water were mixed to make slurry with a dry solids of 30%. The starch slurry was poured into four 500 ml Nalgene bottles and placed in a centrifuge. The floor centrifuge was spun for two minutes at three thousand rpms. The water was poured off and measured with a graduated cylinder. The same volume of DI water was added back to the four containers and the procedure was repeated 5 times. The starch slurry was then tested for Na and Ca concentrations with a Thermo Scientific Orion STAR ISE meter. The sodium level was tested at 30 ppm and a Ca level of 5 ppm. The starch slurry was then pH adjusted with 1M HCl to pH 4.3. The slurry was aliquoted with a pipet boy and transfer pipette into scintillation vials at 10 grams per vial. The falpha-amylase variants were dosed at 4.69 μg Enzyme Protein/gram of starch dry solids.

Liquefaction Procedure:

The heating block controller unit was set to 95 degrees Celsius. The vials were placed in the JKem heating reactor block and the JKem benchtop shaker is set to 25 rpms. A timer is started for two hours. After two hours the vials were taken out of the shaker heater and a 2 ml aliquot from each sample was taken and diluted with 2 ml of DI water.

Data Measurements:

The dry substance C_{dry} was measured using a RFM 340 Refractometer from Bellingham and Stanley. The conductivity a was measured using an Orion conductivity meter and probe model 013005MD. The Osmolality π was measured with an Advanced Instrument Osmometer model 3D3. The constants K1, K2 and K3 are listed below.

| Constants | Symbol | Value | Units | Notes |
|---|---|---|---|---|
| Proportionality | K1 | 45.4 | Osm/kgₛₒₗᵤₜₑ | Proportionality |
| constant 1 | | | | constant |
| | | | | between salt |
| | | | | concentration |
| | | | | and osmolality |
| Proportionality | K2 | 2.16E+06 | μS/cm | Proportionality |
| constant 2 | | | | constant |
| | | | | between salt |
| | | | | concentration |
| | | | | and conductivity |
| Proportionality | K3 | 3.62E+06 | μS/cm | Constant for |
| constant 3 | | | | change in K2 |
| | | | | with different |
| | | | | maltodextrin |
| | | | | concentrations |
| Molecular weight | M_{glucose} | 180 | Dalton | |
| glucose | | | | |

The constants are needed for the equation to measure the corrected Osmolality π (OSM/Kg).

$${\prod }_{corrected} \prod - \frac{{k}_{1} \times σ}{{k}_{2} - ( {k}_{3} \times {C}_{dry} )}$$

Once the corrected Osmolality is calculated (Osm/Kg) π the corrected average Mn molecular weight can now me calculated from the equation below.

$${M}_{n} = \frac{{C}_{dry} \times {10}^{3}}{( 1 - {C}_{dry} ) \times ( \prod - \frac{{k}_{1} \times σ}{{k}_{2} - ( {k}_{3} \times {C}_{dry} )} )}$$

The conductivity corrected dextrose equivalent can also be calculated with the osmolality π, dry substance C_{dry}, and the conductivity σ.

$$DE = {M}_{glucose} \times 0.1 \times ( \prod - \frac{{k}_{1} \times σ}{{k}_{2} - ( {k}_{3} \times {C}_{dry} )} ) \times \frac{( 1 - {C}_{dry} )}{{C}_{dry}}$$

The conductivity corrected DE is reported below in the table. For further details of the method see Y. Rong et al., Determination of dextrose equivalent value and number average molecular weight of maltodextrin by osmometry. (Journal of Food Science, 2009, vol. 74: 1, 33-40).

TABLE 2
pH 4.3, 30 ppm Na, 5 ppm Ca, 2 Hr 95° C. liquefaction

| Substitution | Dextrose equivalent (DE) |
|---|---|
| Reference | 11.2 |
| T181Q | 13.1 |
| H293Q | 13.8 |
| Q393L | 13.6 |
| T116Q | 21.6 |
| T116N | 19.6 |
| T116D | 17.9 |
| T116E | 19.5 |
| T181H | 20.1 |
| S264K (Q264K) | 18.8 |

The below table 3 is included for illustration on how to calculate corrected and uncorrected values of DE and average molecular weight.

TABLE 3
Input

| C_{dry} - sample | σ - Sample | Π - Measured |
|---|---|---|
| dry substance | conductivity | osmolality |
| (gDS/g solution) | (μS/cm) | (Osm/Kg) |
| 0.1734 | 135 | 0.132 |

Output

| Corrected | | Conductivity- |
|---|---|---|
| osmolality (Osm/kg) | Uncorrected DE | corrected DE |
| 0.128 | 11.3 | 11.0 |
| | Uncorrected average | Corrected average |
| | molecular weight (Da) | molecular weight (Da) |
| | 1589 | 1639 |

The uncorrected DE was calculated as:

$$DE = \frac{( 1 - {C}_{dry} ) \times \prod \times {M}_{glucose} \times 100}{{C}_{dry} \times {10}^{3}}$$

The uncorrected average molecule weight was calculated as:

$${M}_{n} = \frac{{C}_{dry} \times {10}^{3}}{( 1 - {C}_{dry} ) \times \prod }$$

### Example 3. Stability Evaluation of T116 Substitutions

Sample Preparation and Incubation:

Purified protein samples were normalized to a concentration of 0.1 mg/ml (100 ppm) in 10 mM K-acetate buffer pH 4.5 containing 0.12 mM Calcium chloride (5 ppm Ca²⁺), 0.01% Triton X-100.

Stress Conditions:

For stressing the protein, normalized protein samples (10 μl, final concentration 5 ppm) were mixed with stress buffer (190 μl containing 100 mM K-acetate pH 4.3, 5 ppm Calcium, 15 ppm Sodium, Triton X-100 and 1.0% cooked starch). After mixing, 50 μl sample was transferred to PCR plate and incubated at 80° C. for 20 min and 50 μl sample (protein+stress buffer) kept at 25° C. for 20 min was considered as unstressed sample.

Activity Assay:

After the incubation period, samples from stressed and unstressed plates were diluted 5× (20 μl sample+80 μl activity buffer containing 100 mM MOPS buffer pH 7.0, 5 ppm Calcium, 15 ppm sodium and 0.01% Triton X-100). To measure the activity, 10 μl each from the diluted sample was transferred into a 384-well plate and to this 40 ul of G7-pNP substrate solution (20 ml of R1 solution and 5 ml of R2 solution, prepared as mentioned in the kit provided by the vendor) was added followed by measurement of kinetics for 10 min at 1 min interval at 405 nm.

Activity of unstressed and stressed sample was determined and the % residual activity was calculated by:

% residual activity=(absorbance of stressed sample/absorbance of Unstressed sample)*100

The improvement factor (IF) was calculated by:

Improvement Factor (IF) of variant=(% residual activity of the variant/% residual activity of the backbone)

Half-life (T½ (in min)) was calculated using the following formulas:

T½(variants)=(Ln(0.5)/Ln(RA-variants/100))*Time

T½(Wild-type)=(Ln(0.5)/Ln(RA-wild-type/100))*Time

TABLE 4
The T116Y substitution clearly provides better
stability than the T116Q substitution.

| Amylase backbone | Modification | T½ (minutes) | IF |
|---|---|---|---|
| SEQ ID NO: 2 | T116Y | 16.85 | 1.20 |
| SEQ ID NO: 2 | T116Q | 13.76 | 1.00 |

1-25. (canceled)

26. An alpha-amylase variant comprising a substitution at a position corresponding to position 116, 393, 181, 293, 264, or 196 of SEQ ID NO: 1, wherein the variant has at least 75%, but less than 100% sequence identity to amino acids 1 to 483 of SEQ ID NO: 1 or amino acids 1 to 481 of SEQ ID NO: 2; and has alpha-amylase activity.

27. The alpha-amylase variant of claim 26, comprising a substitution or combination of substitutions selected from the group consisting of:
T116N, T116D, T116Q, T116E, or T116Y;
L196D;
S264K;
H293Q; and
Q393L;
wherein the variant a residual alpha-amylase activity of at least 45%.

28. The alpha-amylase variant of claim 26, comprising T116D+L196D.

29. The alpha-amylase variant of claim 26, which is isolated.

30. A composition comprising the alpha-amylase variant of claim 26.

31. The composition of claim 30, further comprising an alpha-amylase selected from the group consisting of:
a) an alpha-amylase having at least 90% identity to SEQ ID NO: 3;
b) an alpha-amylase having at least 90% to SEQ ID NO: 4; and
c) an alpha-amylase having at least 90% identity to SEQ ID NO: 5.

32. The composition of claim 30, further comprising a protease.

33. The composition of claim 32, wherein the protease is selected from the group consisting of:
a) a Pyrococcus furiosus protease S having at least 90% identity to the polypeptide of SEQ ID NO: 6;
b) a Thermoascus aurantiacus protease variant, which comprises a combination of mutations selected from the group consisting of:
D79L+S87P+A112P+D142L;
D79L+S87P+D142L; and
A27K+D79L+Y82F+S87G+D104P+A112P+A126V+D142L; wherein the protease variant has at least 90% identity to the polypeptide of SEQ ID NO: 7.

34. A process for producing a syrup comprising:
(a) liquefying a starch-containing material at a temperature above the initial gelatinization temperature in the presence of an alpha-amylase variant of claim 26; and
(b) saccharifying the product of step (a) in the presence of a glucoamylase.

35. The process of claim 34, wherein step (b) is performed in the presence of:
(i) a fungal alpha-amylase;
(ii) an isoamylase; or
(iii) a fungal alpha-amylase and an isoamylase.

36. The process of claim 34, wherein step (b) is performed in the presence of a pullulanase.

37. The process of claim 34, further comprising:
(c) fermenting the product of step (b) using a fermenting organism to produce a fermentation product.

38. The process of claim 37, wherein the fermenting organism is a yeast and the fermentation product is an alcohol.

39. A polynucleotide encoding an alpha-amylase variant of claim 26.

40. A nucleic acid construct or expression vector comprising the polynucleotide of claim 39.

41. A host cell comprising the polynucleotide of claim 39.

42. A method of producing an alpha-amylase variant, comprising cultivating the host cell of claim 41 under conditions suitable for expression of the variant; and optionally recovering the variant.


SEQUENCE LISTING (8 sequences)

SEQ ID NO: 1 - protein, 483 aa, Bacillus licheniformis [sequence omitted]

SEQ ID NO: 2 - protein, 481 aa, Artificial
  Hybrid alpha-amylase
VNGTLMQYFEWYTPNDGQHWKRLQNDAEHLSDIGITAVWIPPAYKAISQADVGYGAYDLYDLGEFWQKGTVRTKYGTKGELQSAIKSLHSRDINVYGDVVINHKAGADATEDVTAVEVDPADRNRVISGEHLIKAWTHFHFPGRGSTYSDFKWYWYHFDGTDWDESRKLNRIYLFQGKTWDWPVSNEFGNYDYLMYADYDYDYPDVVAEITRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPSVFYGDMYGTKGDSQREIPALKHKIEPILKARKQYAYGAQHDYFDHHDIVGWTREGVSSVANSGLAALITDGPGGAKWMYVGRQNAGETWHDITGNRSEPVVINSEGWGEFHVNGGSVSIYVQR

SEQ ID NO: 3 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 4 - protein, 436 aa, Artificial
  Artificial alpha-amylase
MAKYSELEKGGVIMQAFYWDVPSGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 5 - protein, 436 aa, Zea mays
MAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVG

SEQ ID NO: 6 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSS

SEQ ID NO: 7 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 8 - protein, 583 aa, Artificial
  Hybrid alpha-amylase comprising catalytic domain from a Rhizomucor pusillus alpha-amylase fused to a linker and starch binding domain from an Aspergillus niger glucoamylase.
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR